(19) United States
(12) Patent Application Publication
(10) Pub. No.: US 2019/0191964 A1
(43) Pub. Date: Jun. 27, 2019

(54) VASCULAR FORCE REDUCTION SYSTEM

(71) Applicant: COOK MEDICAL TECHNOLOGIES LLC, BLOOMINGTON, IN (US)

(72) Inventors: Georgios Hilas, Bloomington, IN (US); Ralf Spindler, Solsberry, IN (US)

(73) Assignee: COOK MEDICAL TECHNOLOGIES LLC, BLOOMINGTON, IN (US)

(21) Appl. No.: 16/208,794

(22) Filed: Dec. 4, 2018

Related U.S. Application Data
(60) Provisional application No. 62/610,701, filed on Dec. 27, 2017.

(51) Int. Cl. A61B 1/00; A61M 25/00; A61M 25/01

(52) U.S. Cl. CPC A61B 1/0016; A61B 1/00071; A61M 25/0194; A61B 1/00133; A61M 25/0105; A61M 25/0074

(57) ABSTRACT

A system includes a medical device having a proximal end, a distal end, and a section of material between the proximal and distal ends. The system also includes a vascular force reduction system positioned along the section, including at least one support structure supporting at least one rotating assembly, the at least one rotating assembly having a vessel contact surface, wherein the at least one rotating assembly is configured to rotate substantially toward the proximal and the distal ends of the medical device.

### PRIORITY CLAIM

This invention claims the benefit of priority of U.S. Provisional Application Ser. No. 62/610,701, entitled “Vascular Force Reduction System,” filed Dec. 27, 2017, the disclosure of which is hereby incorporated by reference in its entirety.

### BACKGROUND

The present embodiments relate generally to medical devices, and more particularly, to guidewires, catheters, introducers, sheaths, and other devices used in procedures to treat diseased vessels.

In the field of endovascular intervention, physicians treat diseased or damaged vessels using a number of standard procedures. These procedures frequently utilize endovascular devices such as guide wires, catheters, cannulas, sheaths, balloons, stent grafts, etc. Oftentimes, physicians enter the patient's anatomy from a relatively remote location (e.g., the femoral artery), and must navigate instruments through relatively long distances in order to reach the treatment zone. The long distances and tortuous anatomy can require the physician to exert relatively large forces to manipulate the devices.

At the treatment zone and elsewhere, physicians routinely encounter diseased vessels, for example vessels with atherosclerosis and other occlusive diseases. In such diseases, atherosclerotic plaque forms within the walls of the vessel. In occluded areas, it may be difficult to advance or retract endovascular devices, and may be difficult to position the device on a small scale, e.g., millimeters or nanometers. Additionally, because the placement and tracking of endovascular instruments can exert pushing and friction forces against the vessel and plaque, the risk of plaque disruption and downstream embolization can be high.

### SUMMARY

The disclosed embodiments relate to medical devices suitable for use in endovascular procedures. In one aspect, a system may include a medical device with a proximal end, a distal end, and a section of material therebetween. The system may also include a vascular force reduction system positioned along the section. The vascular force reduction system may have at least one support structure supporting at least one rotating assembly that may have a vessel contact surface. The rotating assembly may be configured to rotate toward the proximal and the distal ends.

The rotating assembly may include a bearing and/or a band, such as a band with a traction element. The rotating assembly may include a plurality of rotating elements connected by a band. The section of material of the medical device may include an endovascular device, such as a guidewire, a catheter, an introducer, a sheath, and other device. The support structure may be formed integrally to the section of material, may have a guard, and may include an aperture. The support structure may include a first support structure and a second support structure, which may have a common or different position along a length of the section of the medical device, and/or may have a common or different radial position about the section. The rotating assembly may be configured to rotate within a plane that is approximately coincident or parallel with the section.

In another aspect, a system may include a plurality of rotating assemblies that may be positioned upon a medical device having a section of material positioned between a proximal end and a distal end. Each rotating assembly of the plurality may have a vessel contact surface and may be configured to rotate substantially toward the proximal and the distal ends. The plurality of rotating assemblies may suspend at least a portion of the section within a core protective zone, which may be at least partially bounded by the vessel contact surface of each rotating assembly. The core protective zone may extend along the section from a location of a proximal rotating assembly to a location of a distal rotating assembly. The core protective zone may have a maximum cross sectional dimension of less than approximately 10 mm.

In another aspect, a method may include providing a medical device having a section of material with a proximal end and a distal end and at least one support structure positioned along the section. The support structure may support at least one rotating assembly, which may have a vessel contact surface. The method may further include rolling the vessel contact surface over a vessel wall. The method may further include suspending the section of material in a core protective zone that may be at least partially bounded by the vessel contact surface of at least one rotating assembly.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is an isometric view of a vascular force reduction system-equipped guidewire traversing a plaque deposit within a vessel lumen.

FIG. 2 is a front view of the vascular force reduction system of FIG. 1.

FIG. 3 is a top view of the vascular force reduction system of FIG. 1.

FIG. 4 is a side view of the vascular force reduction system of FIG. 1.

FIG. 5 is an isometric view of another embodiment of a vascular force reduction system.

FIG. 6 is an isometric view of yet another embodiment of a vascular force reduction system.

FIG. 6A is a front view of the vascular force reduction system of FIG. 6.

FIG. 7 is an isometric view of yet another embodiment of a vascular force reduction system.

FIG. 7A is a front view of the vascular force reduction system of FIG. 7.

FIG. 8 is an isometric view of a vascular force reduction system-equipped catheter.

FIG. 9 is an isometric view of another vascular force reduction system.

FIG. 9A is a front view of the vascular force reduction system of FIG. 9.

FIG. 10 is an isometric view of yet another vascular force reduction system.

FIG. 10A is a front view of the vascular force reduction system of FIG. 10.

### DETAILED DESCRIPTION OF THE EMBODIMENTS

In the present application, the term “proximal” refers to a direction that is generally closest to the heart, while the term “distal” refers to a direction that is generally furthest from the heart. The embodiments below are described in connection with endovascular aortic repair procedures, but could also be used for other procedures.

The aorta is the largest artery in the human body and carries blood away from the heart. The size of the aorta normally decreases with distance from the aortic valve in a tapering fashion. The normal diameter of the ascending aorta has been defined as about 21 mm and of the descending aorta as about 16 mm, while the normal diameter of the abdominal aorta is regarded to be less than 30 mm, although the normal range may vary based upon age and sex, as well as daily workload. Raimund Erbel and Holger Eggebrecht, Aortic Dimensions and the Risk of Dissection, 92 Heart Journal 137 (2006). The aortic arch is located distally to the ascending aorta and proximal to the descending aorta. The aortic root is the section of the aorta closest to the heart, and includes the aortic valve and coronary ostia. The right coronary artery and left coronary artery are peripheral vessels in the ascending aorta and circulate blood to the heart tissue itself. Other peripheral vessels near the aortic arch include the brachiocephalic artery, right subclavian artery, right common carotid artery, left common carotid artery, and left subclavian artery. From the aortic arch, the descending aorta continues downward toward the lower limbs. The thoracic aorta is the segment of the descending aorta extending from the aortic arch to about the twelfth vertebrae at the aortic hiatus in the diaphragm, and feeds the bronchial arteries, esophageal arteries, and posterior intercostal arteries, among other branches of the vascular system. The abdominal aorta commences at the distal end of the thoracic aorta and continues downward until it splits into the two common iliac arteries near the fourth lumbar vertebra, supplying the celiac artery, mesenteric arteries, and renal arteries, among other branches. Each common iliac artery subsequently splits into an internal iliac and an external iliac artery. The external iliac artery continues downward until the upper thigh, where it becomes the femoral artery, the primary conduit to carry blood to the lower limbs. For common endovascular procedures, especially aortic repair procedures, physicians often access a patient's vasculature via the femoral artery since it approaches the skin and can be easily palpated. During endovascular procedures, physicians often encounter vessels affected by arteriosclerotic disease.

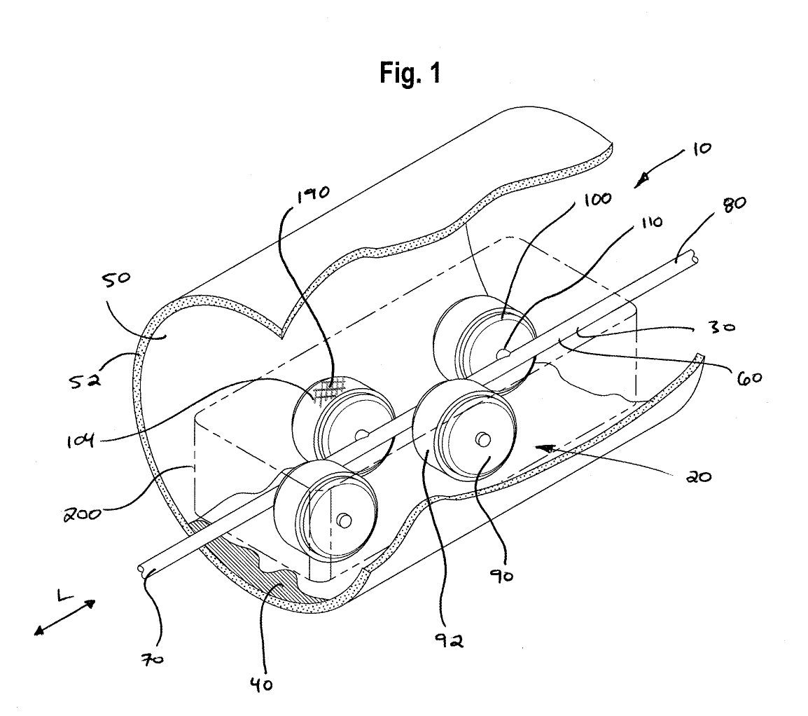

Referring to FIGS. 1-4, an atherosclerotic vessel may be characterized by plaque deposits formed upon the vessel wall, which may occlude the vessel and complicate passage of endovascular devices. Such deposits may be so brittle or unstable that they become detached from the vessel wall when an endovascular device exerts a friction or pushing force upon the deposit. This plaque disruption can be highly dangerous to the patient, for example if it causes downstream embolism.

An endovascular device 10 is equipped with a vascular force reduction system 20. In this embodiment, the endovascular device 10 includes a guidewire 30, although it may alternatively include a catheter, introducer, sheath, or other device. As shown, the guidewire 30 may traverse a plaque deposit 40 within a vessel 50 by “rolling” over it rather than sliding. The endovascular device 10 may have a slender section 60 comprising a material suitable for endovascular procedures, e.g., stainless steel, nitinol, polymeric or other materials, a proximal end 70 that a physician inserts into a patient's vasculature as part of an endovascular procedure, and a distal end 80 that terminates outside the patient's body. The guidewire 30 may generally have a circular cross section, but need not have a circular cross section and need not have the same cross section along its length. However, the cross sectional dimensions of the guidewire 30 should be sufficiently small to permit introduction into a patient's vasculature. The section 60 may be coated with a hydrophilic coating to reduce the input force necessary to move the guidewire 30 in the proximal or distal direction. Additionally or alternatively, the section 60 may be coated with a pharmacological substance, such as heparin or the like.

The force reduction system 20 may include a plurality of rotating assemblies 90 rotatably mounted upon a plurality of support structures 110, such that a vessel contact surface 92 of each rotating assembly 90 may directly or indirectly contact the vessel wall 52 or plaque deposit 40. In different embodiments, the support structure may assume different forms, including branch-type, 8-shape, S-shape, E-shape, aperture-type, and other shapes. The rotating assemblies 90 may be mounted upon the support structures 110 such that each rotating assembly 90 may rotate substantially toward the proximal and distal ends 70, 80 of the endovascular device 10. To enable this rotation, in some embodiments, each rotating assembly 90 may rotate within a plane that is substantially parallel to or coincident with a section of the endovascular device 10. Each rotating assembly 90 may include a rotating element 100 and optionally a band 104. The vessel contact surface 92 of the rotating assembly 90 may be the surface of each rotating element 100 or band 104 that may come into direct contact with a vessel wall 52 or plaque deposit 40 during an endovascular procedure, which may correspond to a radial outermost surface of the rotating element 100 or a band 104. Greater surface area of vessel contact surface 92 may advantageously correspond with lower vessel wall pressures. The plurality of rotating assemblies 90 may be positioned near the proximal end 70 of the endovascular device 10, but may extend distally along a portion of or the entire length of the device 10. In operation, contact between the vessel contact surface 92 and the vessel wall 52 or plaque deposit 40 enables the endovascular device 10 to roll along the vessel wall 52 instead of sliding, for example during an endovascular procedure when a physician manipulates an endovascular device 10 in the proximal or distal directions. The static and dynamic friction forces created by this friction reduction system 20 may be substantially lower than the friction forces created between a vessel wall 52 and the guidewire 30, catheter, introducer, sheath, or other endovascular device 10 not equipped with a vascular force reduction system 20.

Each rotating element 100 of the rotating assembly 90 may include a roller, a bead, a bearing, or other component capable of continuous and reversible revolution within a rotational plane 120 about a revolutionary axis. The rotating element 100 may be generally cylindrical or spherical, but may otherwise include other shapes with approximately constant cross section about the revolutionary axis. The rotating element 100 may have a diameter, a width, an outermost surface 102 (which may correspond to the vessel contact surface 92 in embodiments without bands), and may include a bore capable of receiving an axle 140. Alternatively, the rotating element 100 may include other mounting means to rotatably mount the rotating element 100 upon the support structure 110. Exemplary bearings for the rotating element 100 include No. B0.6-1h26 ball bearings, produced by ISC NSK Micro Precision Co., Ltd. of Tokyo, Japan. The B0.6-1h26 ball bearing features an outside diameter of 2.0 mm, making it dimensionally suitable for endovascular navigation of the femoral artery, along with the iliac artery and aorta, the diameters of which generally exceed that of the femoral artery. See Sandgren, Thomas, et. al, The Diameter of the Common Femoral Artery in Healthy Human: Influence of Sex, Age, and Body Size, 29 Journal of Vascular Surgery, Issue 3, 503-510 (finding a mean common femoral artery diameter of 9.8 mm in male subjects and 8.2 mm in female subjects).

To mitigate sterilization concerns related to the incorporation of rotating assemblies 90 into endovascular devices 10, any rotating assembly 90 may be manufactured with sufficiently tight tolerances to prevent introduction of blood into any internal components, e.g., between a cage and a ball or a roller of a bearing. Additionally, the rotating assembly 90, including the rotating element 100, may be coated with antimicrobial agent(s) to prevent infection or other harmful biological reaction. Like the endovascular device 10 itself, the rotating assembly 90 may be coated with a hydrophilic coating to reduce the input force necessary to move the endovascular device 10 in the proximal or distal direction.

As noted above, each rotating assembly 90 may optionally include one or more bands 104 to enhance vessel wall traction and to reduce pressure exerted upon the vessel wall 52. In such embodiments, each band 104 may completely or partially surround one or more surfaces of the rotating element 100 (e.g., roller, a bead, a bearing, or other component) such that band 104 can make contact with the vessel wall 52 during an endovascular procedure. Thus, in embodiments that include one or more bands 104, a radially outermost surface of the band 104 may correspond with the vessel contact surface 92 of that rotating assembly 90. In some embodiments, one or more rotating elements 100 may include one or more bands 104 snugly fitted to a radial outermost surface 102 of each rotating element 100. In such embodiments, the band 104 may retain its position relative to the corresponding rotating element 100 by friction fit, adhesion, or alternative method. In other embodiments, one or more bands 104 may connect a plurality of rotating elements 100 by running along the radially outermost surface 102 of each rotating element 100; such embodiments may further reduce vessel wall pressures by increasing the surface area in contact with the vessel wall 52.

The band(s) 104 may be constructed from a number of suitable materials, including polymers such as silicone or other suitable materials that provide a high-friction interface with a vessel wall 52. The bands 104 may have a relatively flat cross section with a thickness comparatively small relative to the band width. Alternatively, the bands 104 may have a cross sectional thickness that approaches or exceeds the band width in certain locations, such as a V-shape. In some embodiments, bands 104 may reside within a channel formed into the radially outermost surface 102 of one or more of the rotating elements 100, and may further be located by one or more flanges and/or bead seats. Each band 104 may have a high-friction outer surface 180 that makes direct contact with the vessel wall 52 (i.e., forming the vessel contact surface 92), and the outer surface 180 may feature traction elements 190 capable of reversibly attaching to the vessel wall 52. Ideally, the traction elements 190 will not disturb plaque deposits 40 formed within the vessel 50, for example by exerting relatively large shear forces. One suitable traction element 190 is a synthetic setae configuration, such that the traction element 190 creates a strong retention force with the vessel wall 52, which may be reversed with a relatively small force. Such a traction element 190 configuration is unlikely to disrupt plaque deposits, disrupt the vessel lining, or otherwise damage the vessel or cause vessel trauma. Alternative traction elements may also be suitable, for example ribs, treads, blocks, other raised elements, grooves, and sipes.

The support structure 110 may assume different configurations, for example depending upon whether the vascular force reduction system 20 forms part of a guidewire, a catheter, introducer, sheath, or other endovascular device. Generally, the support structure 110 may include an axle 140 to support each rotating assembly 90, although one axle 140 may support more than one rotating assembly 90. The axle 140 may support the rotating assembly 90 such that the outermost surface 102 of the rotating element 100 or the band 104 can directly or indirectly contact the vessel wall 52 during an endovascular procedure. The axle 140 may be distinct from the guidewire, catheter, or other endovascular device 10, or it may be integral to the device 10. The support structure 110 may enable the rotating assembly 90 to rotate substantially in the proximal and distal directions, e.g., the rotating element 100 may rotate within a rotational plane 120 that is substantially coincident with or parallel to the section 60 of the guidewire, catheter, or other endovascular device 10 upon which the rotating element 100 is mounted. An angle between the rotational plane 120 and the portion of the guidewire, catheter, or other endovascular device 10 may range from zero to about thirty degrees. As the endovascular device 10 navigates tortuous anatomy, the relative positions of the support structures 110 may change. To enable adaptation to dynamic vascular geometry, the support structures 110 may be spaced apart by sufficient distance to enable the endovascular device 10 to flex under bending and torsion forces without one support structure 110 interfering with the operation of another, e.g., by at least approximately 0.1 mm, 2 mm, 3 mm, 4 mm, 5 mm, 10 mm, 15 mm or greater spacing.

Advantageously, the vascular force reduction system 20 protects the endovascular device 10 within a core protective zone 200: an elongate volumetric space bounded by one or more rotating assemblies 90 that protects the portion of the endovascular device 10 supporting the vascular force reduction system 20. For any given embodiment, the core protective zone 200 may have a cross sectional area that approximates a circle, rectangle, or other shape that circumscribes the radial outermost points of the endovascular device 10, e.g., the radial outermost points of one or more rotating assemblies 90. Furthermore, the core protective zone 200 may have a maximum cross sectional dimension that is less than the smallest vascular diameter to be traversed in an endovascular procedure. This maximum cross sectional dimension may vary between embodiments and may further vary based upon the sex, ethnicity, age, and other physiological variable of the intended patient. For example, the maximum cross sectional dimension of the core protective zone 200 may be less than approximately 10 mm, 9 mm, 8 mm, 7 mm, 6 mm, 5 mm, 4 mm, or other dimension depending upon the application.

The core protective zone 200 may begin near the proximal-most rotating assembly 90 and may extend distally along the endovascular device 10 until at least the distal-most rotating assembly 90. The vascular force reduction system 20 suspends a portion of the endovascular device 10 within the core protective zone 200, where one of the rotating assemblies 90—rather than the side of an endovascular device (e.g., an outer surface of guidewire section 60 or a catheter wall)—is likely to make contact with the vessel wall 52 and plaque deposit 40. In operation, as a physician guides the endovascular device 10 through a patient's vasculature, a section of the guidewire, catheter, introducer, sheath, or other device is likely remain within the core protective zone 200, where it is unlikely to make direct contact with the vessel wall 52. Rather, the rotating assemblies 90 contact the vessel wall 52, and the endovascular device rolls along the vessel wall 52 instead of sliding. In this manner, the endovascular devices described herein exert lower forces on the vessel wall, plaque deposits, occlusions, or other portions of the patient's vasculature.

The vascular force reduction system described herein may be constructed in a number of configurations and incorporated into a number of endovascular devices. It shall be understood that technical features of the embodiments illustrated herein may be selectively adapted to different embodiments to suit the needs of a wide range of applications.

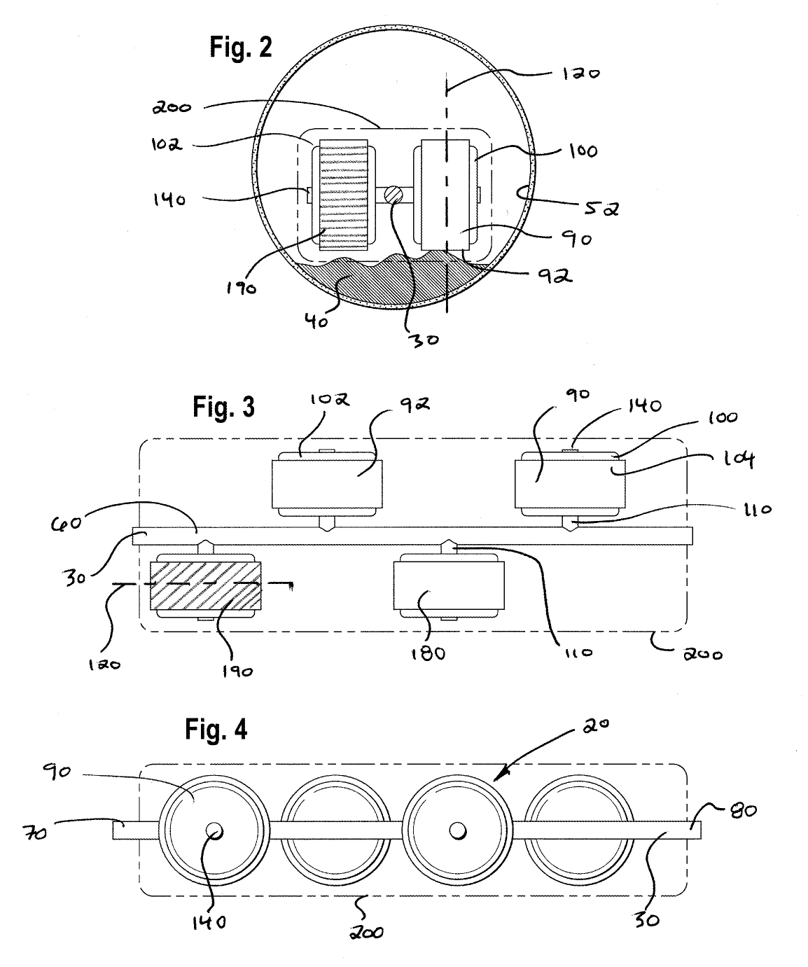

For example, the endovascular device 10 of FIGS. 1-4 includes a guidewire 30 equipped with a vascular force reduction system 20 having a plurality of unguarded, branch-type support structures 110, with each support structure including an axle 140 mounted upon the guidewire 30 in a staggered planar opposed configuration, and with each axle 140 supporting a rotating assembly 90, each of which include a rotating element 100 having an optional band 104 with an outer surface corresponding to the vessel contact surface 92. Each successive rotating assembly 90 resides on approximately the opposite side of the guidewire 30 relative to the next successive rotating assembly 90, with some or all rotating assemblies 90 occupying approximately the same horizontal plane when viewed along the guidewire 30 as shown in FIG. 2. The support structures 110 are spaced apart in order to enable the guidewire 30 to conform to the tortuous anatomy of a patient without one support structure 110 interfering with the operation of another. In the embodiments of FIGS. 1-4 and other embodiments, the support structures 110 may be spaced apart by approximately 0.1 mm to approximately 15 mm, e.g., 10 mm. Each axle 140 may protrude radially outwardly from the guidewire 30 at approximately a ninety degree angle, and may be affixed to the guidewire 30 by any suitable method, for example soldering or welding. In other embodiments, the support structures, including axles, may protrude from the endovascular device at a different angle, for example between approximately sixty to ninety degrees. Each axle 140 may fully extend through the bore of each rotating element 110 or partially therethrough. In either case, the rotating element 100 may be retained upon the corresponding axle 140 by a clip, retainer, cap, detent-notch combination, or similar structure. In this embodiment, to enable contact with the vessel wall 52, the diameter of each rotating assembly 90 may exceed the diameter of the guidewire 30 so that when viewed from the side as in FIG. 4, each rotating assembly 90 projects both above and below the guidewire 30. In this embodiment, the core protective zone 200 has a cross sectional area that approximates a rectangle circumscribing the frontal profile of the rotating assemblies 90 when viewed from the front as in FIG. 2, and has a length that extends from approximately the proximal-most rotating assembly 90 to the distal-most rotating assembly 90. In particular, the vessel contact surfaces 92 of the rotating assemblies 90 may bound the largest cross sectional dimension of the core protective zone 200. In operation, a portion of the guidewire 30 resides within this core protective zone 200, where it is unlikely to contact the vessel wall 52 directly.

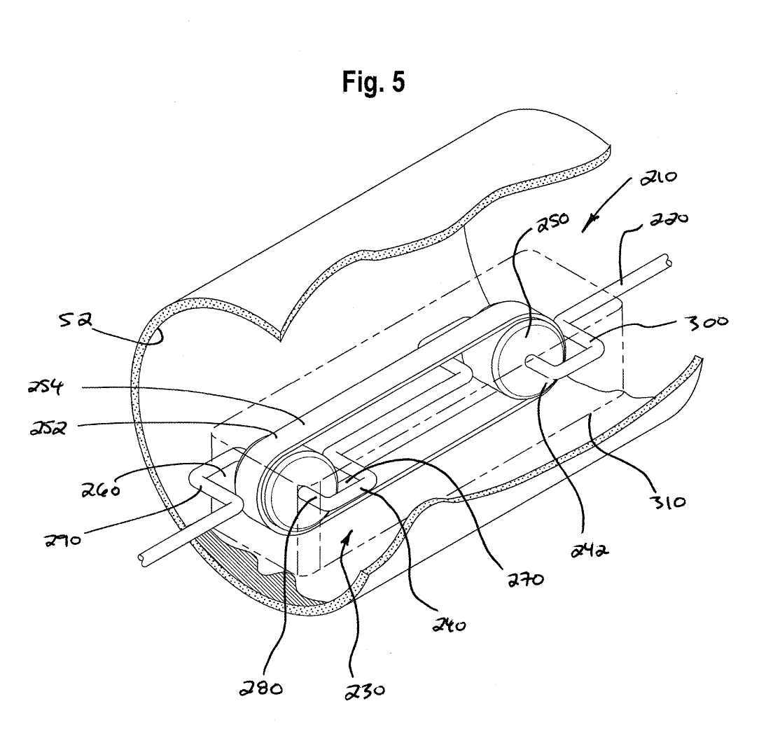

Referring now to FIG. 5, another endovascular device 210 may include a guidewire 220 with a vascular force reduction system 230 having a plurality of S-shaped support structures 240 arranged in a coincident aligned configuration along the guidewire 220. A plurality of rotating assemblies 242 include a plurality of rotating elements 250 that rotate within approximately the same rotational plane as each other, which rotational plane may be approximately coincident with the guidewire 220 for improved tracking, or parallel to the guidewire but separated by a small distance. Other embodiments may utilize different support structures, such as S-type, E-type, or 8-type support structures, to enable rotating elements to rotate within one or more planes that are approximately coincident with the endovascular device. In such other embodiments, each successive support structure may have a different radial orientation than an adjacent support structure, such that each rotating element may rotate within a different rotational plane from adjacent rotating elements. The embodiment of FIG. 5 advantageously offers the potential for a reduced cross-sectional area relative to other configurations. Each support structure 240 may have at least four bends, each with an approximately ninety degree interior angle. The bends of each support structure 240 may form a proximal window 260 and a distal window 270. The central leg of each support structure 240 may serve as an axle 280 for supporting the rotating assembly 242, including the rotating element 250 and a band 252 having a vessel contact surface 254. As the rotating element 250 rotates about the axle 280, it traverses the proximal and distal windows 270, 280. Additionally, a proximal guard 290 and a distal guard 300 may prevent plaque or endovascular debris from fouling operation of the rotating element 250. To reduce vessel trauma in this embodiment and other embodiments with guards, junctions between guards may be smoothed, rounded, blunted, or otherwise formed to avoid point or sharp edges. The elongate band 252 may connect a plurality of the rotating assemblies 242, which may advantageously reduce vessel trauma in operation by distributing forces over the vessel contact surface 254. A vessel contact surface 254 with greater surface area may exert lower pressures on the vessel wall 52, as compared to a vessel contact surface 254 with lesser surface area. A core protective zone 310 may have a frontal cross section that approximates a rectangle or an oval circumscribing the frontal area of the endovascular device 210. Advantageously, the guards 290, 300 further protect a portion of the guidewire 220 within the core protective zone 310.

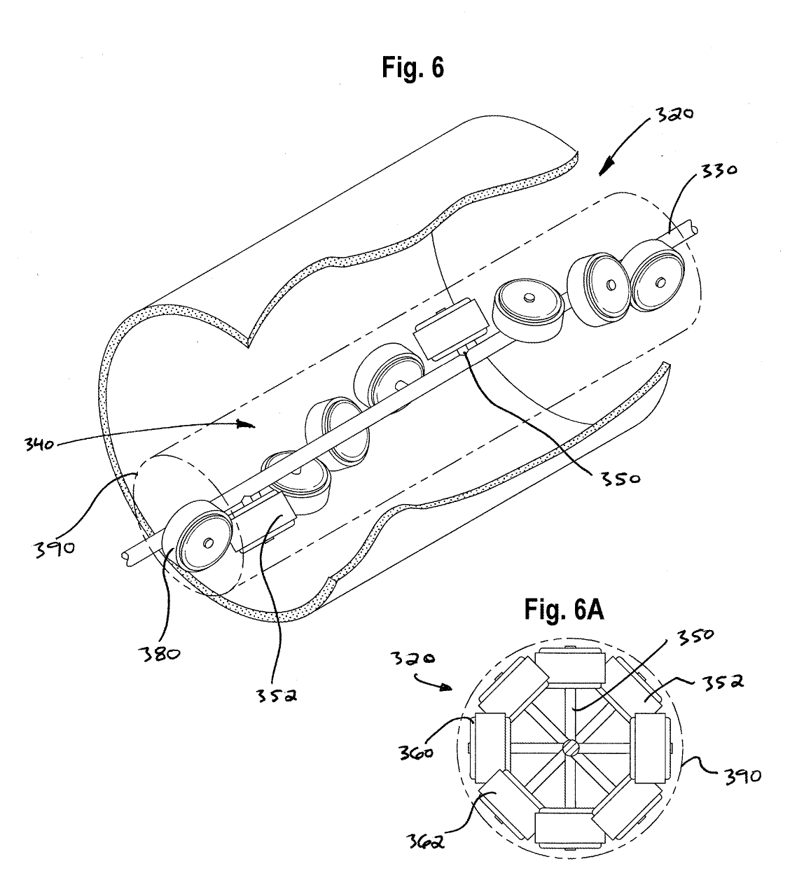

FIGS. 6-6A illustrate another endovascular device 320 including a guidewire 330 and a vascular force reduction system 340 having a helical arrangement of support structures 350 and rotating assemblies 352, each rotating assembly 352 including a rotating element 360 and optionally a band 362. The illustrated embodiment utilizes unguarded, lateral, branch-type support structures 350 that protrude radially outward from the guidewire 330 in a helical series, with each successive support structure 350 protruding at a different angle relative to the adjacent support structures 350. An axle 370 of each successive support structure 350 may have an angle of protrusion that differs by about fifteen to sixty degrees from the axle of the preceding and/or succeeding support structure 350. Successive support structures 350 may be spaced apart by approximately 0.1 mm to approximately 15 mm, e.g., 10 mm, and may be arranged such that an outermost surface of each rotating element 360 or band 362 corresponds to a vessel contact surface 380. When constructed in this configuration, the vascular force reduction system 340 suspends the guidewire 330 within a core protective zone 390 having a cross sectional area that approximates a circle circumscribing the frontal area of the endovascular device 320, as seen in FIG. 6A. Alternative embodiments may incorporate a helical arrangement similar to that illustrated in FIGS. 6-6A, along with different support structures, for example S-type, 8-type, and E-type support structures.

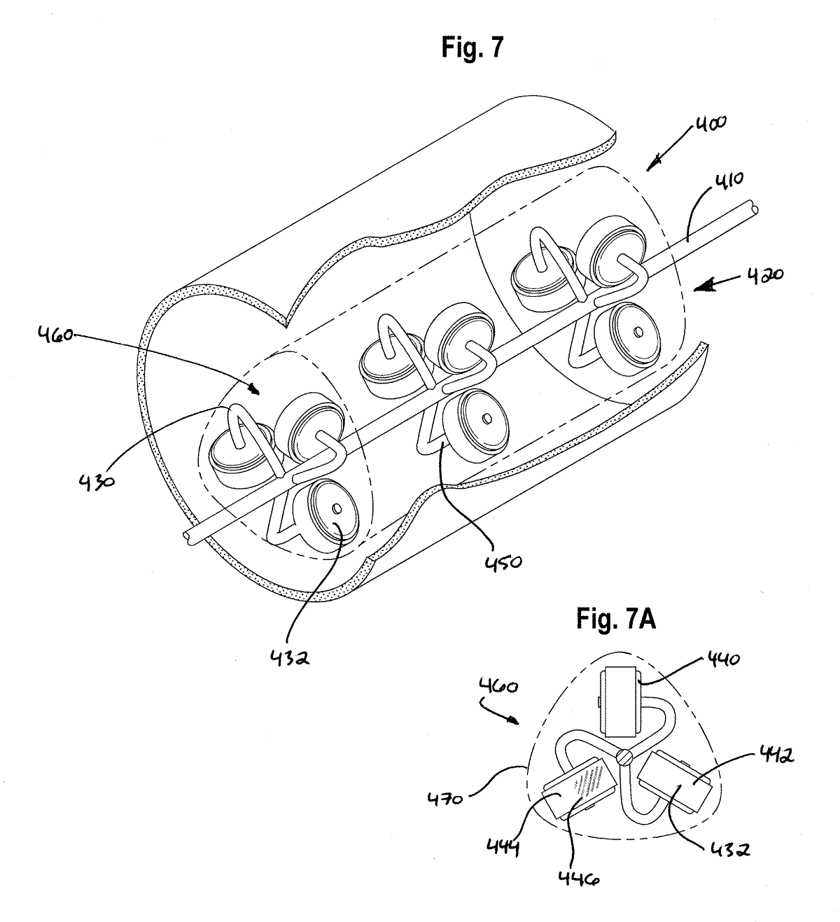

FIGS. 7-7A illustrate another endovascular device 400 including a guidewire 410 and a vascular force reduction system 420 having a star-shaped arrangement of support structures 430 and rotating assemblies 432, each rotating assembly 432 including a rotating element 440 and optionally a band 442. An outermost surface of the rotating element 440 or band 442 may form a vessel contact surface 444, which may have traction elements 446. The support structures 430 and rotating assemblies 432 project radially outward from the guidewire 410, each support structure 430 including an axle 450 that supports the rotating assembly 432. As shown, each support structure 430 may include a continuous segment of material, although each segment may alternatively include two or more joined elements. A group 460 of support structures 430 may reside at a common position along the length of the guidewire 410, and multiple groups 460 may exist along the guidewire 410. Each group 460 may include a plurality of rotating assemblies 432 spaced approximately equally about the circumference of the guidewire 410. Such a group may include at least three rotating assemblies 432 (e.g., with 120 degree radial spacing), although it may be desirable to use a greater number of rotating assemblies 432 (e.g., six rotating assemblies with 60 degree radial spacing). The vascular force reduction system 420 suspends the guidewire 410 within a core protective zone 470 occupying a region extending along the guidewire 410 for the length of the vascular force reduction system 420, with a cross sectional area approximating a triangle or tri-oval circumscribing the frontal area of the endovascular device 400.

Although the foregoing embodiments describe aspects of the invention in the context of a guidewire, the invention is also applicable to other endovascular devices, e.g., catheters, introducers, sheaths, and other similar devices that may directly contact a vessel wall. As with guidewire embodiments, a vascular force reduction system incorporated into a catheter, introducer, sheath, and similar device may be positioned near the proximal end of the device, and may extend distally along a portion of or the entire length of the device. Additionally, in some embodiments, each support structure may be formed integrally to the endovascular device, or formed separately and adjoined thereto. A vascular force reduction system may optionally include one or more bands as described above, for example to enhance vessel wall traction and reduce pressure exerted upon the vessel wall.

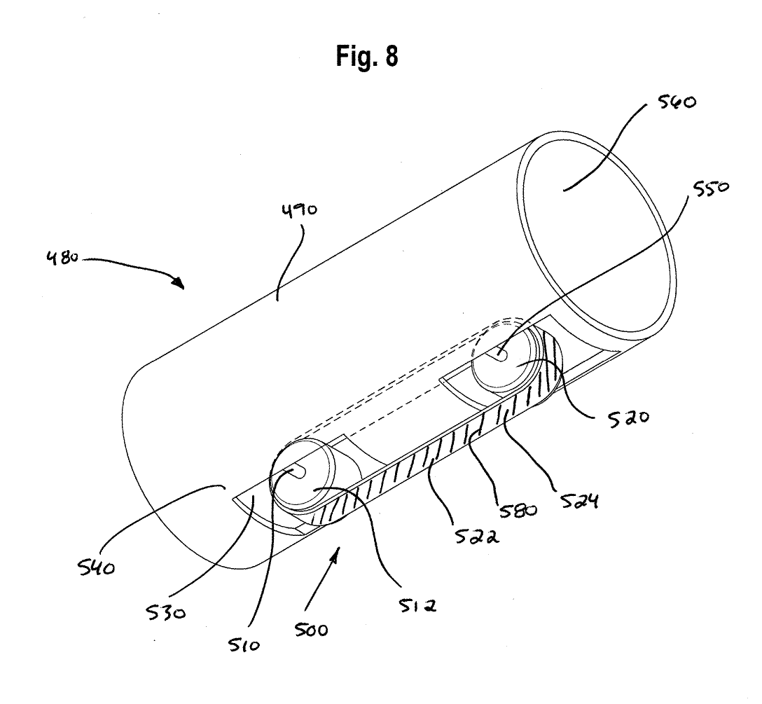

FIG. 8 shows yet another endovascular device 480 including a catheter 490 and a vascular force reduction system 500 utilizing aperture-type support structures 510, wherein a rotating assembly 512 (including a rotating element 520 and a band 522) resides within an aperture 530 extending through a catheter wall 540. An axle 550 may bifurcate each aperture 530 at an orientation that is approximately perpendicular to an elongate section of the catheter 490. The axle 550 may be formed separately and fixed within the aperture 530, or formed integrally to the aperture 530. Integrally-formed axles 550 may include a plurality of sub-axles that project circumferentially inward from the perimeter of the aperture 530. The axle 550 supports the rotating element 520 within the aperture 530 so that it may rotate in substantially the proximal and distal directions. In such a configuration, a portion of each rotating element 520 projects radially outward from the aperture 530, and a portion projects radially inward into a lumen 560 of the catheter. To preserve the ability to insert one or more other medical devices through the catheter lumen 560, it may be advantageous to size the rotating elements 520 and otherwise configure the vascular force reduction system 500 to preserve a passageway within the catheter lumen 560. For example, it may be advantageous if the vascular force reduction system projects into the catheter lumen 560 by a relatively small amount. The band 522 connects the plurality of rotating elements 520 to form a relatively large vessel contact surface 524, and may have traction elements 580 such as synthetic setae. Each aperture 530 guards the rotating element 520 residing within it, so as to protect the rotating element 520 from fouling. In such an embodiment, each rotating element 520 may have a very small diameter, e.g., between approximately 0.1 mm to approximately 5 mm. Also, such an embodiment may advantageously present a smaller cross section because each rotating element 520 is partially recessed within the catheter lumen 560.

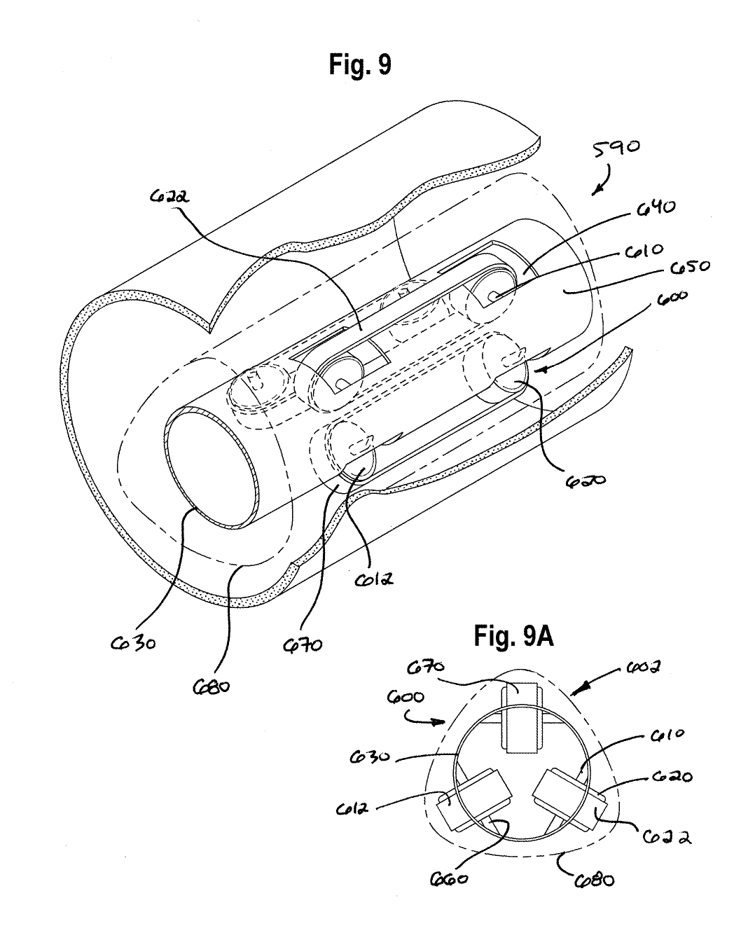

FIGS. 9-9A illustrate another endovascular device 590 including a vascular force reduction system 600 with star-shaped groups 602 of rotating assemblies 612 (including rotating elements 620 and bands 622) positioned along a catheter 630. Like the embodiment of FIG. 8, a plurality of support structures 610 each includes an aperture 640 extending through a catheter wall 650, with an axle 660 that may bifurcate the aperture 640 at an orientation that is approximately perpendicular to a longitudinal section of the catheter 630, and each rotating element 620 may project radially outward through the aperture 640. Each group 602 of rotating assemblies 612 may reside at approximately a common position along the elongate section of the catheter 630, and more than one group 602 may exist along the catheter 630. Within each group 602, the rotational plane of each rotating assembly 612 differs from adjacent rotating assemblies 612. When viewed along the catheter as in FIG. 9A, it can be seen that each group 602 of rotating assemblies 612 may include individual rotating elements 620 spaced approximately equally about the circumference of the catheter 630. Such an arrangement may include at least three rotating elements 620 (e.g., with 120 degree radial spacing), although it may be desirable to use a greater number of rotating elements 620 (e.g., six rotating elements with 60 degree radial spacing). The elongate bands 622 may connect a plurality of rotating elements 620. In operation, each elongate band 622 may reduce vessel trauma by distributing forces over a relative large vessel contact surface 670. In this embodiment, each band 622 connects a plurality of rotating elements 620, although in other embodiments each band may completely or partially surround a single rotating element, or the rotating element may be un-banded. The vascular force reduction system 600 suspends the catheter 630 within a core protective zone 680 occupying a region extending along the catheter 630 for the length of the vascular force reduction system 600, with a cross sectional area approximating a triangle, a tri-oval, or a circle that circumscribes the rotating assemblies 612 when viewed from the front, as in FIG. 9A. Additional pluralities of rotating assemblies 612 may be positioned along the catheter 630 to extend the core protective zone 680.

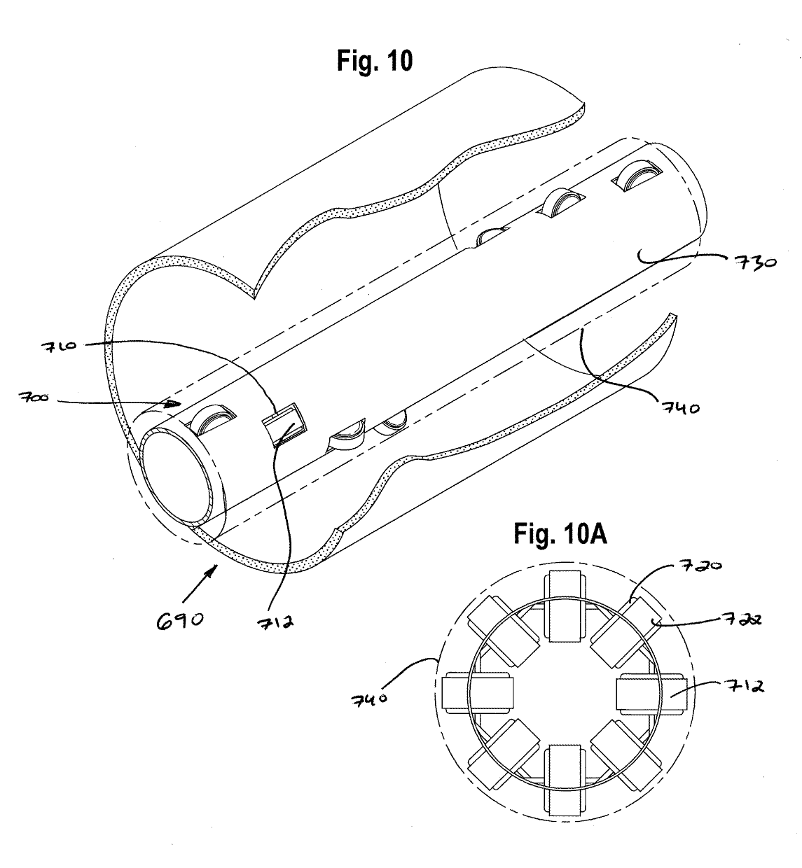

FIGS. 10-10A show another endovascular device 690 incorporating a vascular force reduction system 700 with a helical arrangement of aperture-type support structures 710 and rotating assemblies 712 (including rotating elements 720 and optional bands 722) positioned along a catheter 730. Adjacent rotating assemblies 712 project through a wall of the catheter 730 at different radial angles (angle of protrusion) and at different positions along the catheter 730, so as to form a helix of rotating assemblies 712. Adjacent rotating assemblies 712 may have an angle of protrusion that differs by about fifteen to sixty degrees, and may be spaced apart by approximately 0.1 mm to approximately 10 mm, e.g., 4 mm. The vascular force reduction system 700 suspends the catheter 730 within a core protective zone 740, where the rotating assemblies 712 are likely to make contact with the vessel wall. The core protective zone 740 occupies a region extending along the catheter 730 for the length of the vascular force reduction system 700 and has a cross sectional area that approximates a circle approximately circumscribing the radial outermost surface of the rotating assemblies 712, as seen in FIG. 10A. A similar helical arrangement may be incorporated into other embodiments utilizing different support structures, e.g., branch-type and recess-type support structures. Furthermore, it is possible to incorporate rotating assemblies 712 in a double- or multiple-helix formation (not shown).

It shall be understood that the present embodiments may be constructed in numerous configurations not illustrated herein. For example, in embodiments having a guidewire, the support structures may project from only one side of the guidewire, for example to reduce the frontal profile of the device. In such embodiments, the core protective zone may have a cross sectional area that more closely hews to the side of the rotating assemblies. Additionally or alternatively, embodiments may include U-shaped, V-shaped, trapezoid-shaped, or other polygonal-shaped support structures having legs forming internal bends that together create a window. In such embodiments, one of the legs may operate as an axle for a rotating element, especially if it protrudes from the guidewire at approximately ninety degrees. Thus, the rotating element may traverse the window as it revolves about the axle. Additionally or alternatively, the support structures may occupy the same plane or different planes. In such embodiments, a plurality of support structures may project from approximately the same side of the guidewire, and another plurality may project from another side (e.g., the opposite side), in order to create a core protective zone for the guidewire.

In operation, an operator may insert an endovascular device equipped with a vascular force reduction system into a patient's anatomy, e.g., subcutaneously via an incision. The physician may guide the device in the proximal direction, for example to a position proximal of an aneurysm. During such a procedure, the operator may roll the endovascular device over the vessel wall or plaque deposit. In particular, the vessel contact surface of the vascular force reduction system will roll over the vessel wall or deposit. Depending on the type of endovascular device, the physician may guide one or more additional endovascular devices over or within the vascular force reduction system-equipped device. For example, the physician may guide a catheter over a guidewire, and one or both of the catheter and guidewire may be equipped with a vascular force reduction system. The physician may ultimately remove the endovascular device equipped with the vascular force reduction system from the patient's vasculature, e.g., by withdrawing it in the distal direction.

The foregoing endovascular devices equipped with vascular force reduction systems may advantageously exert lower radial and shear forces on the vessel wall, reducing the risk of vessel damage during endovascular procedures. Similarly, by rolling over plaque deposits instead of sliding in an uncontrolled manner, endovascular devices incorporating a vascular force reduction system may exert lower radial and shear forces on plaque deposits, thereby reducing embolism risk. Furthermore, by reducing input forces, the vascular force reduction systems disclosed herein may enable devices to navigate tortuous anatomy that might otherwise be unnavigable with endovascular devices known in the art. Separately, because endovascular devices equipped with a vascular force reduction system may require lower input forces, those devices need not have the stiffness or rigidity that would otherwise be necessary; as a result, endovascular devices can be manufactured with reduced cross sectional areas, which further reduces trauma to the patient. Together, these advantages may enable a greater number of patients to qualify for endovascular procedures who might otherwise be ineligible, for example due to highly tortuous or occluded vasculature.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

We claim:

1. A system, comprising:
a medical device comprising a proximal end, a distal end, and a section of material between the proximal and distal ends; and
a vascular force reduction system positioned along the section, comprising at least one support structure supporting at least one rotating assembly, the at least one rotating assembly having a vessel contact surface,
wherein the at least one rotating assembly is configured to rotate substantially toward the proximal and the distal ends of the medical device.

2. The system of claim 1, wherein the at least one rotating assembly comprises a bearing.

3. The system of claim 1, wherein the at least one rotating assembly comprises a band.

4. The system of claim 2, wherein the band comprises a traction element.

5. The system of claim 2, wherein the at least one rotating assembly comprises a plurality of rotating elements connected by the band.

6. The system of claim 1, wherein the section of material comprises an endovascular device.

7. The system of claim 1, wherein the section of material comprises a guidewire.

8. The system of claim 1, wherein the section of material comprises a catheter.

9. The system of claim 1, wherein the at least one support structure is formed integrally to the section of material.

10. The system of claim 1, wherein the at least one support structure comprises a guard.

11. The system of claim 1, wherein the at least one support structure comprises an aperture.

12. The system of claim 1, wherein the at least one support structure comprises a first support structure and a second support structure, wherein the first and second support structures have a common position along a length of the section.

13. The system of claim 1, wherein the at least one support structure comprises a first support structure and a second support structure, wherein the first and second support structures have different radial positions about the section.

14. The system of claim 1, wherein the at least one rotating assembly is configured to rotate within a plane that is approximately coincident with the section.

15. The system of claim 1, wherein the at least one rotating assembly is configured to rotate within a plane that is approximately parallel to the section.

16. A system, comprising:
a plurality of rotating assemblies positioned upon a medical device having a section of material positioned between a proximal end and a distal end, each rotating assembly of the plurality having a vessel contact surface and being configured to rotate substantially toward the proximal and the distal ends,
wherein the plurality of rotating assemblies suspends at least a portion of the section within a core protective zone at least partially bounded by the vessel contact surface of each rotating assembly.

17. The system of claim 15, wherein the core protective zone extends along the section from a location of a proximal rotating assembly of the plurality to a location of a distal rotating assembly of the plurality.

18. The system of claim 15, wherein the core protective zone has a maximum cross sectional dimension of less than approximately 10 mm.

19. A method, comprising:
providing a medical device comprising a section of material having a proximal end and a distal end, at least one support structure positioned along the section, the at least one support structure supporting at least one rotating assembly, and the at least one rotating assembly having a vessel contact surface; and
rolling the vessel contact surface over a vessel wall.

20. The method of claim 19, further comprising suspending the section of material in a core protective zone that is at least partially bounded by the vessel contact surface of the at least one rotating assembly.
